(19) United States
(12) Patent Application Publication
(10) Pub. No.: US 2008/0097785 A1
(43) Pub. Date: Apr. 24, 2008

(54) System and method to quantify patients clinical trends and monitoring their status progression

(75) Inventor: Walid Ali, Croton-On-Hudson, NY (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/571,370

(22) PCT Filed: Jun. 30, 2005
(86) PCT No.: PCT/IB05/52188

§ 371 Date: Dec. 28, 2006

Related U.S. Application Data
(60) Provisional application No. 60/584,201, filed on Jun. 30, 2004.

(51) Int. Cl. G06F 19/00; A61B 5/02; G06Q 50/00

(52) U.S. Cl. 705/2; 600/300

(57) ABSTRACT

A system for facilitating identification of correlations over time between patient monitoring signal histories to facilitate the making and revising of healthcare decisions includes patient monitoring equipment (144), a memory (146), a computing device (148), and a display device (152). A method for facilitating identification of correlations over time between patient monitoring signal histories to facilitate the making and revising of healthcare decisions includes designating (158) a time frame, providing (160) two patient monitoring signal histories over the time frame, constructing (162) a three-dimensional geometric surface model of the signal histories over the time frame, and visually displaying (164) the model to facilitate visual identification of correlation between the signal histories.

This disclosure relates to patient monitoring. More particularly, this disclosure relates to correlating multiple patient monitoring signals. Still more particularly, this disclosure relates to representing such signals collectively as a geometric construct to facilitate such correlating and further cross analysis.

Providing patients with healthcare typically includes monitoring various signals related to aspects of a patient's condition, including a variety of internal and external events and states, such as pulse, temperature, and blood pressure, other biological activity, intake of medication, timing of medication, among others.

Healthcare professionals monitor, and make healthcare decisions based at least in part, on such signals. In addition, in order to make the best decisions possible, healthcare professionals often prefer to review prior signal values as part of their monitoring. Often, in reviewing prior signal values, healthcare professionals identify correlations between signal values. The healthcare professional can then modify patient care as appropriate based on the correlations identified. The term “correlation,” as used herein, means relatedness of a signal to at least one other signal. The term “trend,” as used herein, means a correlation in which at least one of the signals is a time signal where the signal has an overall consistent behavior, e.g., increasing or decreasing trend.

In critical care cases, healthcare providers operate under significant pressure. An example of such a case is where a patient undergoes a septic shock in the course of treatment. Quick and correct treatment is often required to save patients' lives or avoid serious health consequences. In such cases, it is especially important that healthcare providers quickly and accurately identify correlations in reviewing the prior signal values.

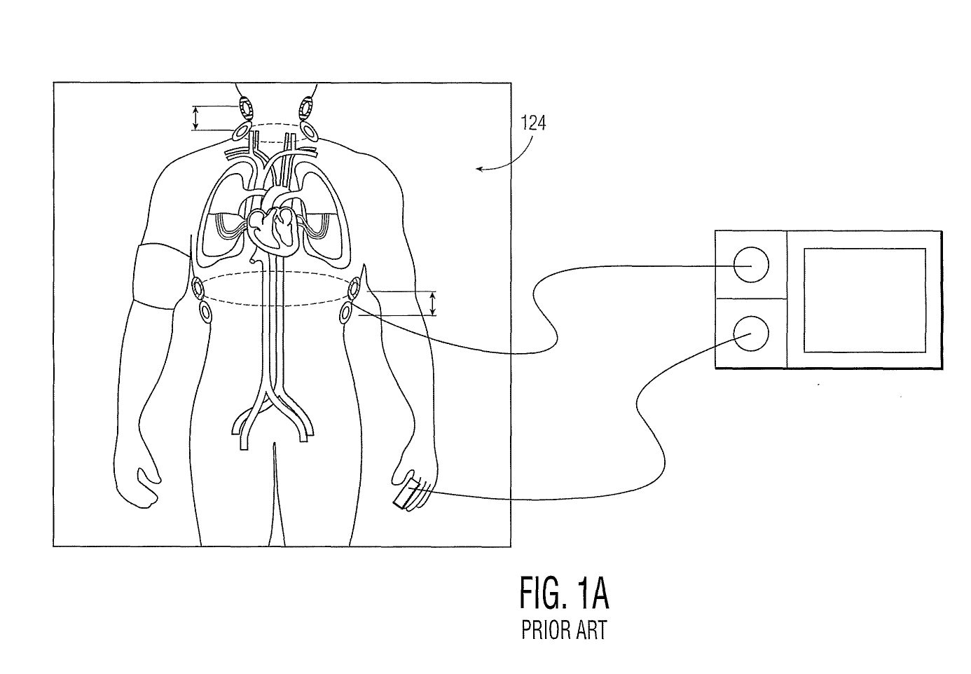

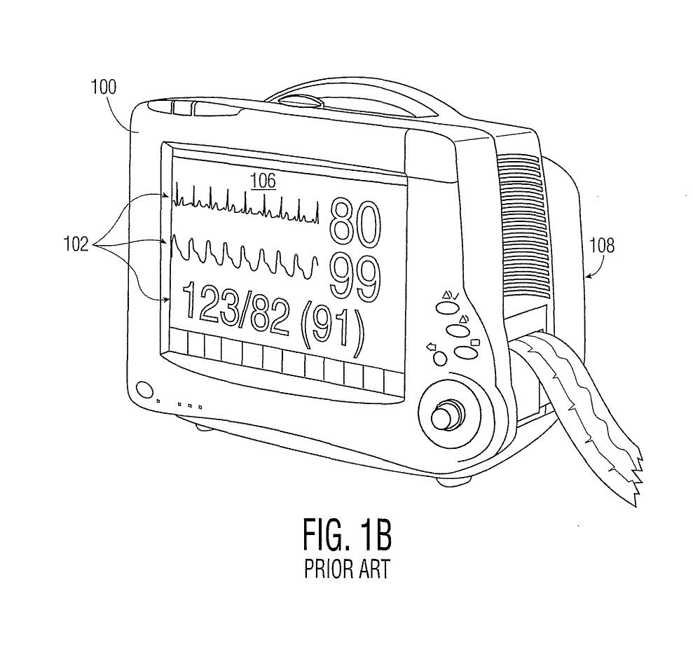

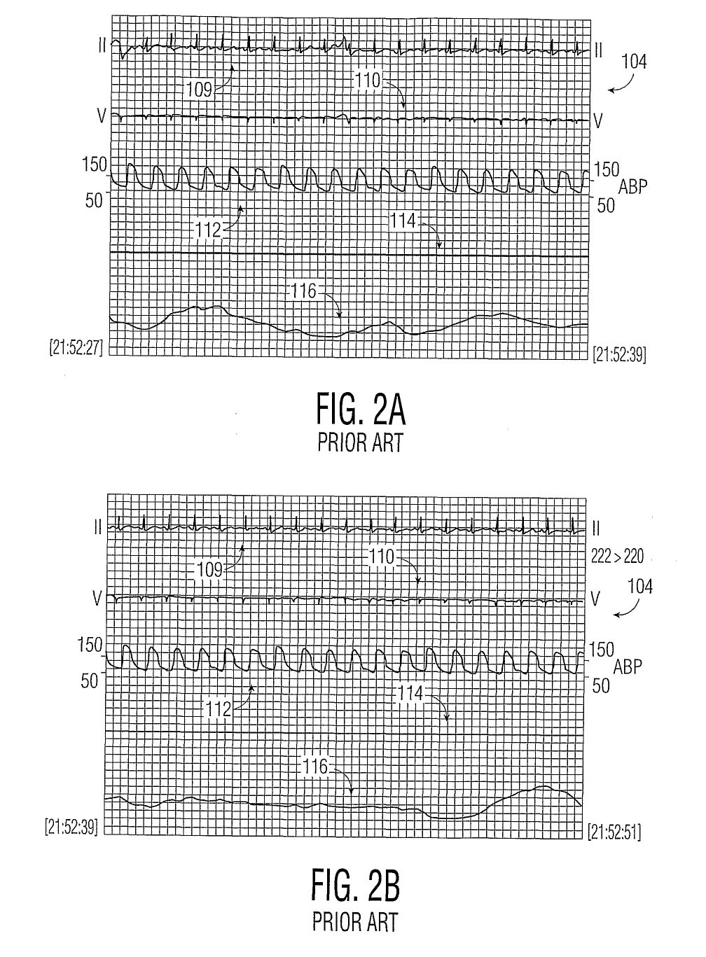

Patient monitoring devices provide current and prior signal values to healthcare professionals via a variety of conventional methods. FIG. 1 depicts such a prior art device 100 in use, presenting signal values and waveforms 102 corresponding to the patient's condition. FIG. 2 presents a series of waveforms 104 such as are commonly used in the prior art to represent signals.

Auditory alarms can be used and commonly indicate that specific signal values are no longer being detected or have gone beyond a predetermined range. However, auditory alarms provide very limited information and do not typically convey information about prior signal values.

Visual displays, such as liquid crystal displays, are also common. Visual displays can present current and prior signal values to healthcare providers in numerical, tabular, and graphical format, among others. However, visual displays limit the quantity of information that healthcare providers can consider to one or a limited number of displays. The limited quantity of information can prevent a healthcare provider from quickly identifying correlations. Moreover, the presentation format forces the healthcare provider to mentally assimilate all of the presented information, which takes time and, especially in time-pressure situations, jeopardizes the accuracy of the conclusions due to easily incurred human error.

Printing devices can provide current signal values and commonly provide prior signal values. One advantage of printouts showing prior signal values is that a very large volume of information can be clearly presented. However, sorting through such a large volume of material takes a significant amount of time and, like reviewing visual displays, requires the healthcare provider to mentally assimilate all of the relevant information to identify correlations; however, attempting to mentally assimilate such a very large amount of information under time-pressure conditions introduces a significant chance for human error.

What is clearly needed is a method and system for representing a history of multiple patient monitoring signals in a way that allows a healthcare professional to easily, quickly, and accurately review the patient's corresponding clinical status and clinical history.

This disclosure provides such a method and system. These and other advantages, as well as additional inventive features, will be apparent from the present disclosure.

For a more complete understanding of the present disclosure, and the advantages thereof, reference is now made to the following brief descriptions taken in conjunction with the accompanying drawings, in which like reference numerals indicate like features.

FIG. 1 shows a PHILIPS MP 30 INTELLIVUE™ patient monitoring device.

FIG. 2 presents a series of waveforms corresponding to patient monitoring signals.

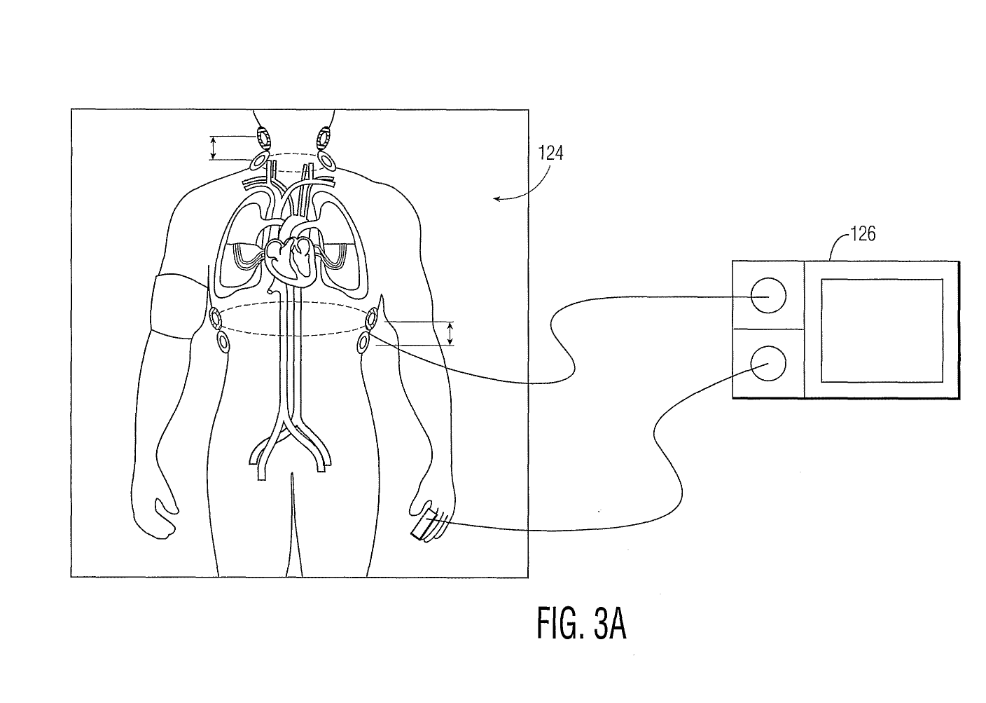

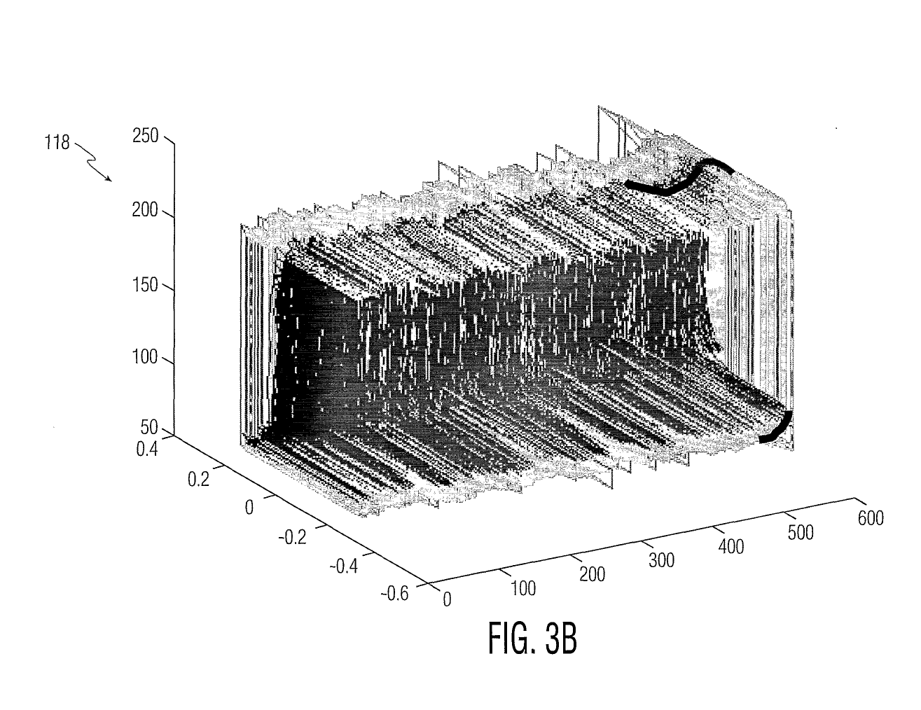

FIGS. 3-6 illustrate a three-dimensional (3D) geometric surface constructed from the signals of FIG. 2 and a corresponding time signal.

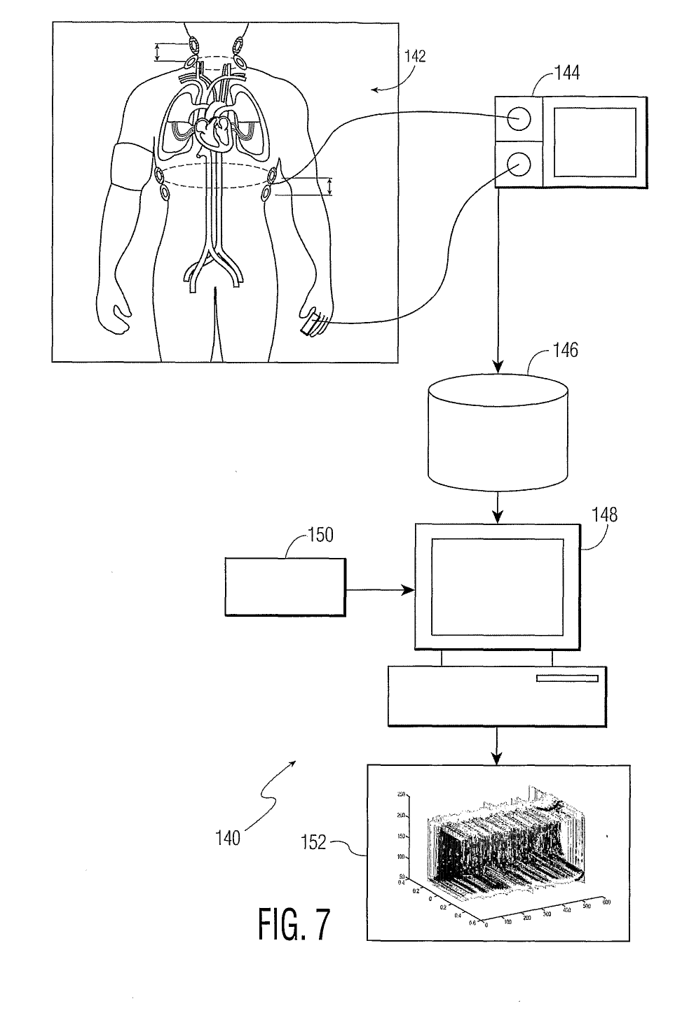

FIG. 7 displays an overview of a system for capturing and displaying patient monitoring signal histories using a 3D graphical surface representation.

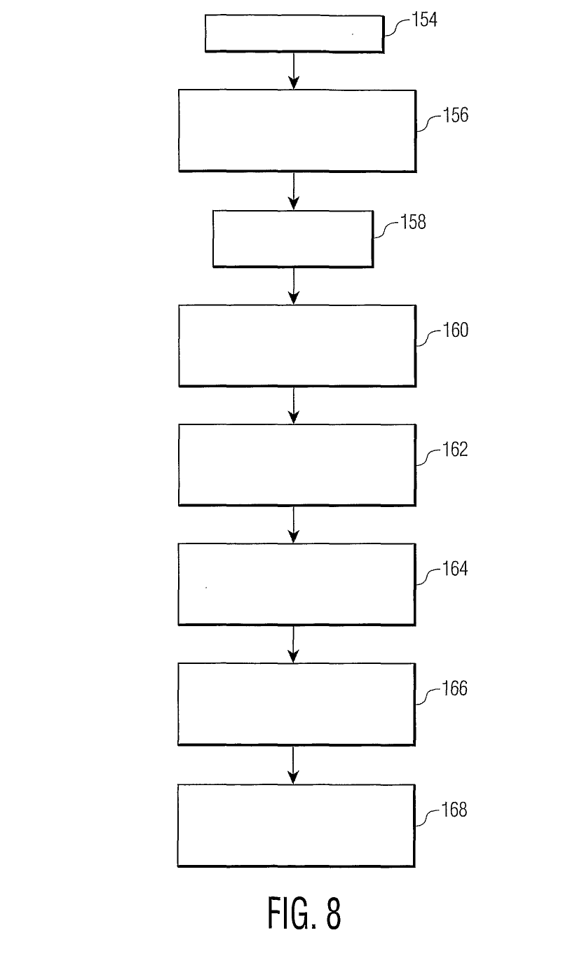

FIG. 8 shows a process for achieving a 3D graphical surface representation of patient signal histories.

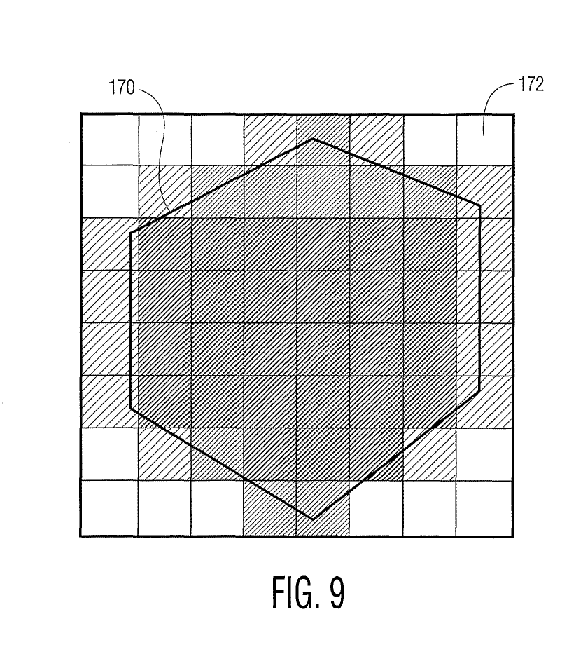

FIG. 9 shows a voxel footprint having a greater than one-to-one voxel-to-pixel correspondence.

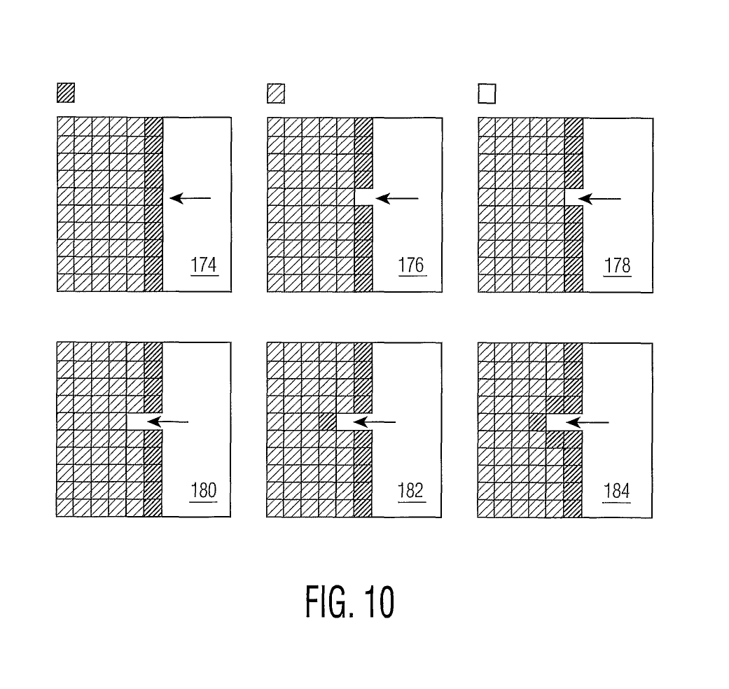

FIG. 10 depicts removal of a surface voxel and the corresponding update of the surface list.

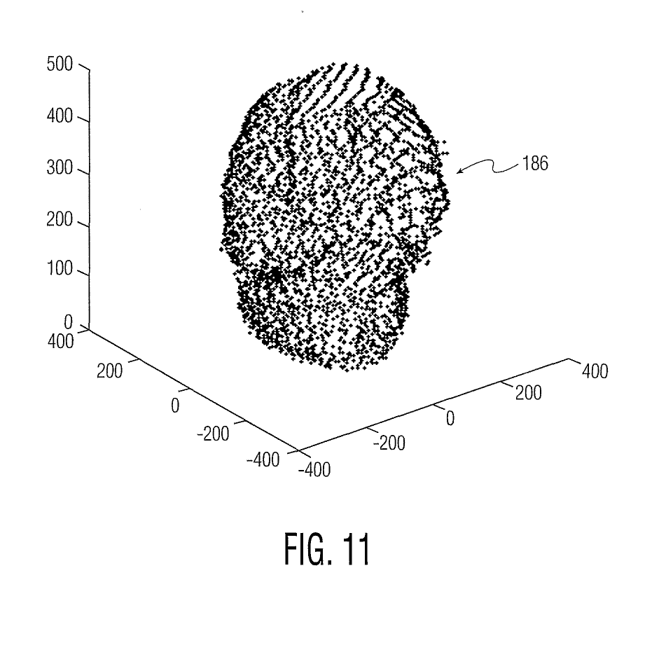

FIG. 11 depicts scattered data points on a brain surface.

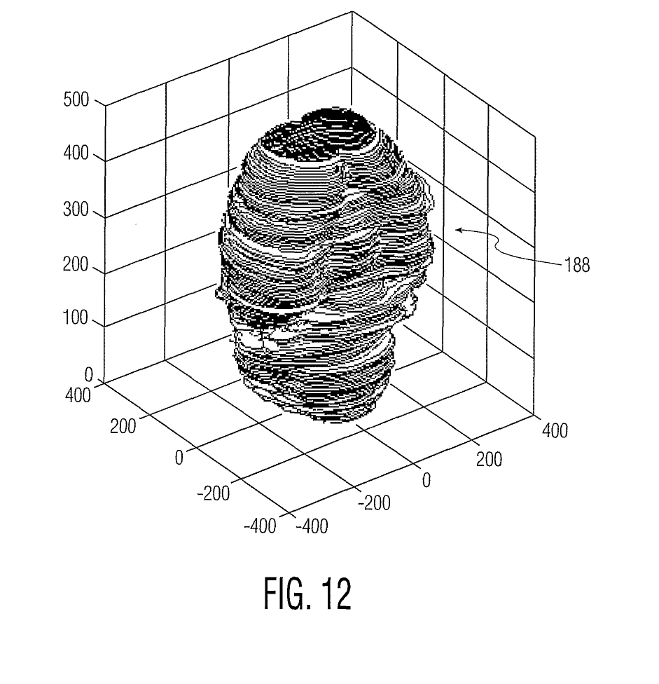

FIG. 12 depicts a B-spline surface fitted to the scattered data points of FIG. 11.

This disclosure provides a system for facilitating identification of correlations over time between patient monitoring signal histories to facilitate the making and revising of healthcare decisions, including patient monitoring equipment 144, a memory 146, a computing device 148, and a display device 152. This disclosure also provides a method for facilitating identification of correlations over time between patient monitoring signal histories to facilitate the making and revising of healthcare decisions, including the steps of designating 158 a time frame, providing 160 two patient monitoring signal histories over the time frame, constructing 162 a three-dimensional geometric surface model of the signal histories over the time frame, and visually displaying 164 the model to facilitate visual identification of correlation between the signal histories.

Other aspects, objectives and advantages of the invention will become more apparent from the remainder of the detailed description when taken in conjunction with the accompanying drawings.

The method and system taught by this disclosure allow a healthcare provider to easily, quickly, and accurately review the reaction of a patient's monitored (e.g., hemodynamic and echocardiogram) signals to external stimuli, such as medication, as well as internal stimuli, such as cardiac arrhythmia. Such a history of signal value correlations-based analysis provides the healthcare provider with information which is essential in understanding and navigating the patient's treatment course.

FIG. 1 shows a PHILIPS MP 30 INTELLIVUE™ patient monitoring device 100. The device 100 includes a visual display 106 capable of displaying up to four waveforms 102 and a printing module 108 capable of producing paper documentation of signal values.

IntelliVue MP30 patient monitors provide monitoring capability and measurements. Such monitors can operate on a networked platform using wireless or wired communication technology. The IntelliVue MP30 includes an integrated 10.4-inch color SVGA display capable of displaying three or four waveforms. Up to three invasive blood pressures and two temperatures can be tracked, and the IntelliVue MP30 includes an integrated recorder, which is capable of printing out waveforms or tabular information for later review.

FIG. 2 presents a series of waveforms 104 corresponding to hemodynamic signal monitoring of the II and V ECG leads 109 and 110, ambulatory blood pressure (ABP) 112, pleth 114, and respiration signals 116. These signals will be utilized for purpose of example in creating a corresponding geometric construct 118 as described in connection with FIGS. 3-6. The method and system taught by this disclosure accommodate equally well other signals. For example, an embodiment of the present invention could accommodate EEG, pulse, temperature, and any other measurable biological activity.

Returning to FIG. 1, the IntelliVue MP30 works with a multi-measurement server module which interfaces with patient monitoring equipment to enable monitoring of multiple internal and external events and states associated with a patient's condition. The server module is capable of storing up to eight hours of patient monitoring signal history data.

The results of the method and system taught by this disclosure can be presented through a device such as that shown in FIG. 1. Therefore, one embodiment of the present 100 invention would include a module attachable to the device shown in FIG. 1 in order to embody the method and system taught by this disclosure.

It is well known that people absorb information visually much more quickly and easily than through reading—hence the well known saying, “A picture is worth a thousand words.” For that reason a graphical representation of a patient monitoring signal history is a more effective mechanism for conveying information than a tabular list of values presented in numeric format. This is the reason for the widespread use of waveforms to represent signals corresponding to aspects of the patient's condition.

Likewise, a single graphical representation containing information corresponding to two patient monitoring signal histories over time and showing correlations between the two histories over time is more effective for conveying that information than two separate graphical representations (e.g., waveforms) of the histories in which any correlation must be identified by manually aligning numerically or spatially identified index values.

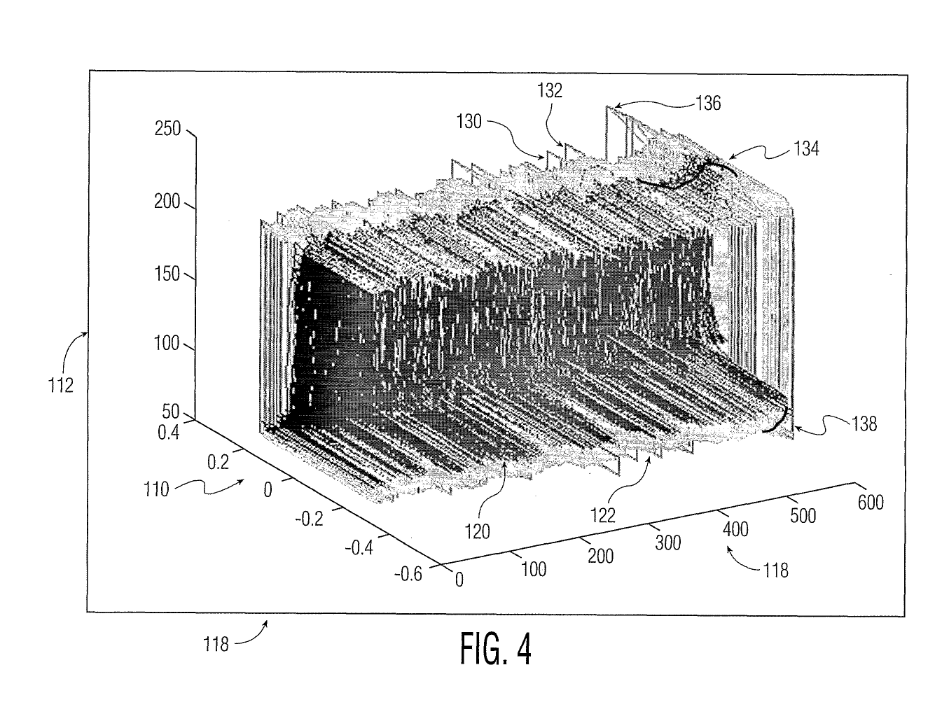

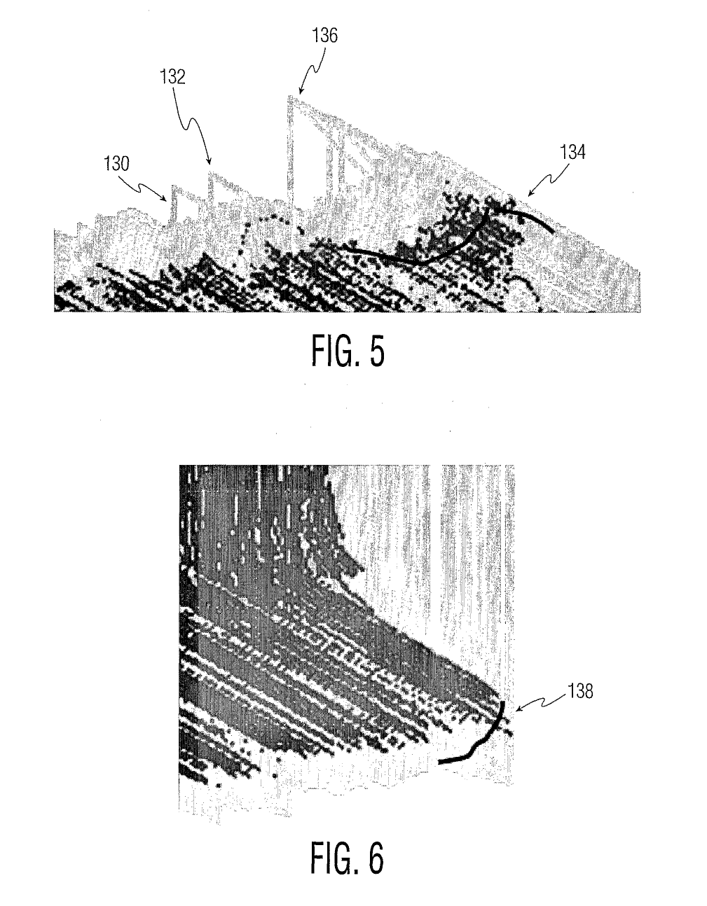

FIGS. 3-6 illustrate a three-dimensional (3D) geometric surface 120 constructed from two hemodynamic signals of FIG. 2 and a corresponding time signal. A surface modeled 120 on hemodynamic data point triplets is shown, as is an encapsulating rectangular mesh 122 of the surface 120. FIG. 3 depicts a patient 124 being monitored using patient monitoring equipment 126 which is adapted to graphically present the resultant 3D geometric surface 120. FIG. 4 shows the resultant surface 120 in greater detail, while FIGS. 5 and 6 show two pertinent areas of the graphical presentation in greater detail.

Three axes are utilized to depict signal data points with reference to time 128, ambulatory blood pressure (ABP) 112, and secondary electrocardiogram lead (V ECG lead) 110. A geometric surface 120 is constructed based on these three signals to facilitate visualization and perception by fitting a 3D surface to the data point triplets defined by three signal histories. Any means of constructing a 3D surface to represent the correlations between the data point triples will be suitable, and an explanation of several approaches is given below.

Spikes 130 and 132 in the ABP signal and an increase in the heart rate, which reflects an attempt by the sympathetic system to regulate blood pressure. The first part of the curve 134 corresponds to a drop in ABP, which supports this interpretation.

The sympathetic system's increase of the electric activities of the heart is evidenced by an increase in ECG amplitude 136. Responsive changes in ABP are evidenced 134, including a slight increase followed by a slight decrease. Finally, patient stabilization is indicated by value stabilization 138 of the ECG and ABP signal values.

FIG. 7 displays an overview 140 of a system for capturing and displaying patient monitoring signal histories using a 3D graphical surface representation. Two aspects of a patient's 142 condition are monitored by patient monitoring equipment 144. The resultant patient monitoring signal histories are stored in a patient monitoring signal history database 146. A computing device 148 with 3D graphics capability pulls desired signal history data corresponding to user parameters, e.g., time frame, which have been input 150. The computing device 148 generates a corresponding 3D geometric surface representation of the pulled data, and provides that representation to a device for displaying 152 the 3D surface representation.

FIG. 8 shows a process for achieving such a representation. A patient is monitored 154, and the resulting patient monitoring signal histories are stored 156 in a signal history database. User parameters are input 158, as for example, by using controls communicably coupled to a computing device configured to access the signal history database. The signal history database is accessed and two signal histories are retrieved 160 from the database, in accordance with the user parameters. A 3D geometric surface representing the signal histories over time is constructed 162 and displayed 164 to the user. The user visually identifies 166 medically significant correlations between the signal histories over time and makes a healthcare recommendation, decision, or revision after considering such correlations 168.

The 3D geometric surface presentation enables the healthcare provider to easily, quickly, and accurately discern important correlations between patient monitoring signal histories so they may be considered in recommending, deciding, or revising the patient's course of treatment.

Steinbach, E., Girod, B., Eisert, P., Betz, A.,“3-D object reconstruction using spatially extended voxels and multi-hypothesis voxel coloring”, IEEE 15^{th} international conference on pattern recognition, Vol. 1, pp. 774-777, 2000 (STEINBACH) provides an illustration fitting a 3D surface to data point triplets together with a survey of other methods.

One class of 3D model acquisition techniques contains techniques to construct a 3D surface model of an object by registering depth maps from two or more views of the object. Another class of 3D model acquisition techniques contains techniques to construct a 3D surface model of an object by computing the intersection of outline cones, which back project the object's silhouette from all available views.

A third class of 3D model acquisition techniques combines aspects of each of the above-described classes, and contains techniques to construct a 3D surface model of an object by coloring volume elements (voxels) by comparing the color of corresponding pixels when the voxel is viewed from various angles.

Voxels can be projected into the image plane to a single point. Contrast this with “extended voxels” which are projected into the image plane with a small footprint—possibly allowing coverage of more than one pixel by a single voxel. For example, FIG. 9 shows a voxel footprint 170 having a greater than one-to-one voxel-to-pixel correspondence. This is caused by the voxel's size, its cubical shape, and the perspective view of the figure. The degree of shading in each pixel 172 corresponds to the percentage of that pixel covered by the voxel's footprint 170. Volume is discretized in all three dimensions so the object can be represented by a set of voxels, each being associated with a data point triplet. Initially, all voxels are transparent.

The kth voxel's color is defined by the following equation:

Hₗₘₙ^{k}=(R(Xᵢ, Yᵢ), G(Xᵢ, Yᵢ), B(Xᵢ, Yᵢ))

where H(k, lmn) is the voxel's color hypothesis, (l, m, n) is the voxel's data point triplet, (Xi, Yi) is a data point pair representing the pixel position corresponding to the voxel center (xl, ym, zn) projected into the ith camera view, and R, G, and B are color components. Furthermore,

$${X}_{i} = - {f}_{x} \frac{{x}_{li}}{{z}_{ni}} , {Y}_{i} = - {f}_{y} \frac{{y}_{m i}}{{z}_{ni}} , \\ {with \\ ( {x}_{li} , {y}_{mi} , {z}_{ni} )}^{T} = {{R}_{i} ( {x}_{l} , {y}_{m} , {z}_{n} )}^{T} + {T}_{i} .$$

where Ri is the object's rotation in ith view and Ti is the object's translation in the ith view. The camera geometry and scaling relating pixel coordinates to world coordinates are represented by fx and fy. The following represents a condition for associating H(k, lmn) with a voxel V(lmn):

$$\vert \frac{{R}_{i} ( {X}_{i} , {Y}_{i } )}{{N}_{i} ( {X}_{i} , {Y}_{i } )} - \frac{{R}_{j} ( {X}_{j} , {Y}_{j} )}{{N}_{j} ( {X}_{j} , {Y}_{j} )} \vert + \vert \frac{{G}_{i} ( {X}_{i} , {Y}_{i } )}{{N}_{i} ( {X}_{i} , {Y}_{i } )} - \frac{{G}_{j} ( {X}_{j} , {Y}_{j} )}{{N}_{j} ( {X}_{j} , {Y}_{j} )} \vert + \vert \frac{{B}_{i} ( {X}_{i} , {Y}_{i } )}{{N}_{i} ( {X}_{i} , {Y}_{i } )} - \frac{{B}_{j} ( {X}_{j} , {Y}_{j} )}{{N}_{j} ( {X}_{j} , {Y}_{j} )} \vert < ⊖ \\ with$$

$${N}_{i} ( X , Y ) = {R}_{i} ( X , Y ) + {G}_{i} ( X , Y ) + {B}_{i} ( X , Y ) .$$

Alternately, robustness can be improved by increasing the threshold value for heavily occluded voxels by about 50% by modifying the above condition as follows:

$${⊖}_{new} = ( \frac{3}{2} - \frac{1}{2} \frac{{F}_{n}}{{F}_{o}} ) ⊖$$

FIG. 10 depicts removal of surface voxels and corresponding updates of the surface list as follows:

A surface voxel is selected 174;

A surface voxel is removed 176;

The surface is updated 178 as the newly exposed voxel is converted from an invisible voxel to a surface voxel;

The newly converted voxel is removed 180;

A newly exposed voxel immediately behind the removed converted voxel is converted 182 to a surface voxel; and

Other voxels newly exposed by the removal of the first converted voxel are themselves converted 184 to surface voxels.

Fernand S. Cohen, Walid S. Ibrahim Ali, and Chuchart Pintavirooj, “Ordering and Parameterizing Scattered 3D Data for B-Spline Surface Approximation” IEEE tanrs. PAMI, May 2002 describes several approaches to constructing a geometric surface to model a set of data points, including the preferred approach of using B-splines.

One approach to surface representation is based on Extended Gaussian Image (EGI) surface representation using pentagonal or triangular cells. However, the approach encounters many-to-one mapping issues in representing non-convex surfaces.

Wavelets can also be used to represent surfaces. Wavelets provide a simple hierarchical structure, and techniques for the numerical analysis of wavelets are well-developed.

Another approach to surface representation utilizes quad-trees, which decompose 2D regions iteratively into successively smaller quadrants. Oct-trees provide an analogous technique for representing 3D surfaces by decomposing 3D regions iteratively into successively smaller cubic cells. Oct-trees tend to require a significant amount of information to describe objects of greater than minimal complexity and tend to result in lost information.

A symmetrical axis transform (SAT) technique can be used to represent 2D and 3D regions. In practice, 2D objects are represented using maximal disks within the object, while 3D objects are represented using maximal spheres within the object.

Yet another approach, called “distance profile,” the surface is decomposed into distance contours, each being the loci of all points on the surface at a fixed distance from a point called the “center point” of the contour. The critical point is sensitive to noise, but the method is invariant to surface rotations and translations.

B-spline representation involves the use of parametric models to construct a smooth surface that “best” fits a set of scattered unordered 3D range data points. B-spline is well suited for surface representation because it possesses continuity, affine invariance, and local-shape controllability. Parameters needed for B-spline surface construction as well as finding the ordering of the data points can be calculated based on the geodesics of the surface's extended Gaussian map. A set of control points can be analytically calculated by solving a minimum mean square error problem for best surface fitting. The set of scattered unordered 3D range data points can be obtained from any source: for example, a structured light system (a range finder); point coordinates on the external contours of a set of surface sections, as for example in histological coronal brain sections; or other source.

Walid S. Ibrahim Ali and Fernand S. Cohen, “3D Geometric Invariant Alignment of Surfaces with Application in Brain Mapping”, proc IEEE conf. Computer vision and pattern recognition, CVPR 1999 describes an approach to the problem of full or partial alignment of surfaces in the presence of affine transformations, local deformation, and noise. FIG. 11 depicts scattered data points 186 on a brain surface. FIG. 12 depicts a B-spline surface fitted 188 to the scattered data points 186 of FIG. 11.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing embodiments of the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. For example, an embodiment could include a system configured to display a continuously updated 3D geometric surface representation of two signal histories as the histories are generated in real time by patient monitoring equipment. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A automatic method for identifying time correlations between patient monitoring signal histories, which time correlations provide data sufficient to render and/or revise healthcare decisions, the method comprising the steps of:
designating (158) a time frame within which said time correlations are identified;
providing (160) two patient monitoring signal histories over the time frame;
identifying (166) correlations between the two signal histories;
constructing (162) a three-dimensional geometric surface model of the signal histories over the time frame; and
visually displaying (164) the model to facilitate visual identification of correlation between the signal histories.

2. The method of claim 1, wherein the step of providing the two signal histories further comprises the step of:
retrieving (160) the signal histories from a signal history database.

3. The method of claim 2, further comprising the step of:
receiving (158) user parameters, and wherein the step of retrieving the signal histories further comprises the step of:
retrieving (160) the signal histories from the signal history database in accordance with the user parameters.

4. The method of claim 1, further comprising the steps of:
monitoring (154) two aspects of a patient's condition;
generating one of the two signal histories based on one of the two aspects; and
generating the other of the two signal histories based on the other of the two aspects.

5. The method of claim 5, wherein the step of monitoring one of the aspects of the patient's condition includes one of:
a) monitoring the patient's intake of a medication;
b) monitoring the patient's pulse.

6. The method of claim 1, further comprising the step of:
recommending (168) healthcare based on the correlations.

7. The method of claim 1, wherein the step of constructing the model comprises the step of:
modeling the two signal histories over time using B-splines.

8. The method of claim 1, wherein the step of constructing the model comprises the step of:
plotting data point triplets in a three-dimensional Cartesian coordinate system having:
a first axis corresponding to the magnitude of the first patient monitoring signal;
a second axis corresponding to the magnitude of the second patient monitoring signal; and
a third axis corresponding to time.

9. The method of claim 1, further comprising the step of:
constructing an encapsulating rectangular mesh (122) based on the three-dimensional geometric surface model (120).

10. A system for facilitating identification of time correlations between patient monitoring signal histories, which correlations facilitate the making and revising of healthcare decisions, the system comprising:
patient monitoring equipment (144) which monitors two aspects of a patient's condition, generates two patient monitoring signal histories based on said two monitored aspects and stores the two signal histories in a memory (146) communicably coupled to the patient monitoring equipment (144);
a computing device (148) communicably coupled to the memory (146) for retrieving the two signal histories from the memory (146) and generating a three-dimensional geometric model representing the two signal histories over time;
a display device (152) communicably coupled to the computing device (148) and adapted to visually display the model.

11. The system of claim 10, further comprising:
an input device communicably coupled to the computing device to accept user parameters (150), transmit the parameters to the computing device (148), wherein the computing device (148) is adapted to retrieve the two signal histories from the memory (146) based on the user parameters (150).

12. The system of claim 11, wherein the input device is a keyboard.

13. The system of claim 12, wherein the computing device is adapted to generate the model using B-splines.

14. The system of claim 10, wherein the computing device is adapted to generate the model by plotting data point triplets in a three-dimensional Cartesian coordinate system having:
a first axis corresponding to the magnitude of the first patient monitoring signal;
a second axis corresponding to the magnitude of the second patient monitoring signal; and
a third axis corresponding to time.

15. The system of claim 10, wherein the computing device is adapted to generate an encapsulating rectangular mesh (122) based on the three-dimensional model (120).

16. A system for generating a graphical representation of correlations over time of a number of patient monitoring signal histories, said graphical representation constructed by the system to automatically provide a complete clinical review of the patients' clinical history and status, as indicated by same signal history correlation, comprising:
a patient monitoring apparatus capable of displaying at least two signal waveforms over a given time frame, therefore defining at least two signal histories;
a computing device in communication with the patient monitoring apparatus capable of identifying correlations within the at least two signal histories, and constructing a three-dimensional geometric surface model using the signal histories over said time frame wherein said correlations are easily identified.

17. The system of claim 16, wherein the at least two signals are represented in 3D to depict signal points as a function of time.

18. The system of claim 16, further comprising a memory for storing patient monitoring signal histories.

19. The system of claim 16, further comprising a display for visually communication said graphical representation.

20. The system set forth in claim 16, further comprising a user input device in order to allow manual entry of parameters.
